(19) United States
(12) Patent Application Publication
(10) Pub. No.: US 2008/0139702 A1
(43) Pub. Date: Jun. 12, 2008

(54) Use of Fatty Alcohols as Plasticizer to Improve the Physical-Mechanical Properties and Processability of Phb and its Co-Polymers

(75) Inventors: Wanderson Bueno De Almeida, Sao Jose dos Campos (BR); Pablo Silva Bizzari, Jacarei (BR); Nazareno Antonio Sertori Durao, Brodowski (BR); Jefter Fernandes Do Nascimento, Sao Paulo City (BR)

Correspondence Address: COGNIS CORPORATION;PATENT DEPARTMENT, 300 BROOKSIDE AVENUE, AMBLER, PA, 19002, US

(73) Assignee: PHB INDUSTRIAL S.A., Serrana (BR)

(21) Appl. No.: 11/573,278

(22) PCT Filed: Aug. 6, 2004
(86) PCT No.: PCT/EP04/08874

§ 371 Date: Sep. 25, 2007

(51) Int. Cl. C08K 5/05; C12P 7/02

(52) U.S. Cl. 524/14; 524/381; 524/47

(57) ABSTRACT

A plasticized composition including (i) at least one biopolymer selected from the group consisting of poly hydrobutyrate (PHB) and PHB copolymers; and (ii) a plasticizer containing (a) a saturated or unsaturated, linear or branched C₆₋₃₀ fatty alcohol, and (b) a glycerol ester of a linear or branched, saturated or unsaturated C₆₋₂₄ fatty acid, wherein (a) and (b) are present in a ratio of 100:0 to 75:25 by weight is provided.

### CROSS-REFERENCE TO RELATED APPLICATIONS

This application is a national phase filing under 35 U.S.C. §371 and claims priority to International Application No. PCT/EP2004/008874 which has an International filing date of Aug. 6, 2004, and which designated the United States of America, the entire disclosure of which is hereby incorporated herein by reference.

### BACKGROUND OF THE INVENTION

1. Field of the Invention

The invention relates generally to compositions including at least one biopolymer and a plasticizer, and more specifically, relates to compositions including at least one polyhydroxybutyrate (PHB) biopolymer or PHB copolymer, a plasticizer containing a C₆₋₃₀ fatty alcohol, and optionally a glycerol ester of a C₆₋₂₄ fatty acid.

2. Background Information

Today, worldwide and industrially, is known the need to produce biodegradable and biocompatible materials employing renewable raw materials and energy sources, through environmentally non-aggressive processes. On the market, the more successful biodegradable biopolymer applications are disposable materials like, for example, agrochemical and cosmetics packaging, and medicinal applications.

An important biodegradable biopolymer family is Polyhydroxyalcanoates (PHAs). They are polyesters made by many microorganisms through natural synthesis. There are more than 170 microorganisms in the literature, and the commercial advantage of the PHAs is not only linked to the biodegradable qualities, but also to the thermo-mechanical properties and low production costs.

The most representative PHAs are: PHB (poly-3-hydroxybutyrate), PHB-V (poly(hydroxybutyrate-co-hydroxyvalerate)), P4HB (poly-4-hydroxybutyrate), P3HB4HB (poly(3-hydroxybutyrate-co4-hydroxybutyrate)) and any PHA mcl (medium chain length) polyhydroxyalcanoates, and PHHx (polyhydroxyhexanoate) is a typical biopolymer of this family. The PHAs chemical structure can be described as a polymeric chain made up of the repeating unit below:

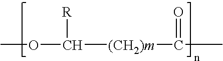

wherein R is a variable length chain alkyl group. M and n are integral numbers, and in the polymers mentioned above, R and M have the following values:
PHB: R═CH3, m=1
PHB-V: R═CH3 or CH3-CH2-, m=1
P4HB: R═H, m=2
P3HB-4HB: R═H or CH3, m=1 or 2
PHHX: R═CH3-CH2-CH2-, m=1

Many of the PHAs can be processed on extruders by common injection molding without too many modifiers required for good processability. Also, there is a possibility to process these polymers in cast and coating film systems for applications such as food industrial packs.

Depending on the development level these polymers can be used to make thin packs at high-speed discharge for personal hygiene articles. Even where intrinsically the biodegradable properties are required, the PHAs have made technical and commercial application aspects very clear, like compost packs, golf tees, fishing articles and other things made of plastic materials that may be left outdoors.

In the agro business, PHAs can be used to fabricate flowerpots, reforesting tubes, ground coating films and principally, in controlling release systems for nutrients, herbicides, insecticides and others.

For biomedical applications, PHAs can be used for microencapsulating compounds controlling biodegradation and absorption of medical sutures and osseous fracture fixation pins. The great developments in natural science in the last two decades, especially in biotechnology, have permitted the use of the many microorganisms, natural or genetically modified, in the commercial production of PHAs.

Although many applications have been made with the bacterial cells “in natura” (without the PHAs solvent agent), like moldable materials, as explained in U.S. Pat. No. 3,107,172, the PHAs commercial applications, in the most cases required high purity levels for good plastic properties. The utilization of solvents is crucial for the PHA extraction and recuperation of the residual biomass for an adequate processability purity level.

In EPA-01455233 A2 are described some procedure possibilities for the digestion of cells with a PHA aqueous suspension, using enzymes or surfactant agents for non-PHA substance solubilization. This patent shows, with reference to the solvent extraction process, possible limitations because of the elevated production costs. However, if an elevated purity product is desired, the solvent step isn't eliminated.

In an organic solvent extraction process, frequently cited in the literature for PHA extraction and recuperation of bacterial biomass, partially halogenated hydrocarbons solvents are utilized, such as chloroform (U.S. Pat. No. 3,275,610), ethanol/methanol chlorine (U.S. Pat. No. 3,044,942), chloroethane and chloropropane with the boiling point between 65 to 170° C., 1,2-dichloroethane and 1,2,3-trichloropropane (EP0014490 B1 and EP2446859).

Other halogenated solvents, like dichloromethane, dichloroethane and dichloropropane are cited in U.S. Pat. No. 4,562,245 (1985), U.S. Pat. No. 4,310,684 (1982), U.S. Pat. No. 4,705,604 (1987) and European patent 036.699 (1981) and German patent 239.609 (1986).

Biopolymer extraction and purification processing of biomass by employing halogenated solvents is absolutely prohibitive today. They are extremely harmful to human health and the environment. Therefore, a solvent for PHA extraction and purification must be in the first place, environmentally friendly.

Therefore, the use of resources damaging to the environment in any production step must be avoided. Also the energy source used in the production process must come from a renewable source. Otherwise it is senseless to have a low environmental impact plastic; if in your production only non-renewable resources are utilized, for example. A very interesting approach for this problem is the incorporation of the bioplastic productive chain for agro industry, particularly for the sugar and alcohol industry (Nonato, R. V., Mantelatto, P. E., Rossell, C. E. V., “Integrated Production of Biodegradable Plastic (PHB), Sugar and Ethanol”, Appl. Microbiol. Biotechnology, 57:1-5, 2001).

U.S. Pat. No. 6,127,512 discloses a polyester pellet composition comprising a polyhydroxyalkanoate (PHA) having a molecular weight (Mw) of greater than about 470,000 and a plasticizing quantity of at least one plasticizer selected from the group consisting of:
A. high boiling point esters selected from
phthalates and isophthalates of the formula: [FIG. 1] where R1 is C₁₋₂₀ alkyl cycloalkyl or benzyl; (ii) citrates of the formula: [FIG. 2] where R1 is hydrogen or C₁₋₁₀ alkyl, and R2 is C₁₋₁₀ alkyl, C₁₋₁₀ alkoxy or C₁₋₁₀ alkoxyalkyl;
adipates of the formula R1 —O—C(O)—(CH₂)₄—C(O)—OR2 where R1 and R2 which may be the same or different are C₂₋₁₂ alkyl or C₂₋₁₂ alkoxyalkyl;
sebacates of the formula R1 —C(O)—(CH₂)₈—C(O)—O—R1 where R1 is C₂₋₁₅ alkyl or C₂₋₁₅ alkoxyalkyl;
azelates of the formula R1 —O—C(O)—(CH₂)₇ —C(O)—R1 where R1 is C₂₋₁₂ alkyl, benzyl, or C₂₋₁₂ alkoxyalkyl;

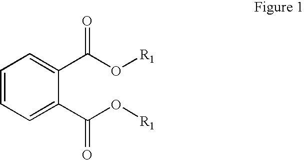

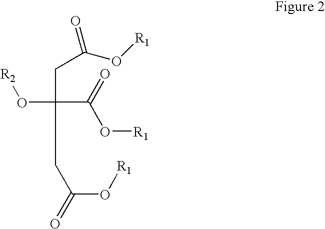

B. alkyl ethers/esters of the formula R2 —(O)—CH₂—CH₂)ₙ —O—R1 where R1 is alkyl or —C(O)-alkyl, R2 is alkyl and n is 2 to 100; or where R1 is hydrogen and either: R2 is alkylphenyl where the alkyl is C₂₋₁₂ alkyl, and n is 1 to 100; or R2 is CH₃—(CH₂)₁₀—C(O)— and n is 5, 10, or R2 is CH₃—(CH₂)₇—CH═CH—(CH₂)₇—C(O)— and n is 5 or 15;
C. epoxy derivatives of the formula CH₃—(CH₂)n-A-(CH₂)n —R in which the A is an alkene containing one or more double bonds (i.e., unsaturated fatty acids), n is 1 to 25 and R is C₂₋₁₅ alkyl; or epoxy derivatives of triglycerides containing one or more double bonds per fatty acid chain with chain lengths from C₆₋₂₆.
D. substituted fatty acids selected from the group consisting of sorbitan monolaurate, sorbitan monooleate, poly(oxyethylene)(20) Sorbitan monolaurate, poly(oxyethylene)(4)lauryl ether, and butyl acetyl ricinoleate; and
E. polymeric esters of the formula —O—C(O)—R1-C(O)—O—R2-O— in which R1 and R2 are both independently C₂₋₁₂ alkylene, or R2 may be derived from a diol.

Other patents connected with this above patent are: WO9923146A1 and AU1281499A1.

### SUMMARY OF THE INVENTION

Briefly described, a plasticized composition includes (i) at least one biopolymer selected from the group consisting of poly hydrobutyrate (PHB) and PHB copolymers; and (ii) a plasticizer containing (a) a saturated or unsaturated, linear or branched C₆₋₃₀ fatty alcohol, and (b) a glycerol ester of a linear or branched, saturated or unsaturated C₆₋₂₄ fatty acid, wherein (a) and (b) are present in a ratio of 100:0 to 75:25 by weight.

### DETAILED DESCRIPTION OF THE INVENTION

Fatty alcohols with or without glycerol fatty esters are used as plasticizers in PHB and its co-polymer compositions to improve the processability and physical-mechanical properties. The plasticizers are incorporated in the PHB and its co-polymers by mixing in a dry blend system.

It is an object of the present invention to provide plasticizer compositions for PHB and its co-polymers, to improve the physical/mechanical properties of the processed PHB and its co-polymers. PHB is defined as a Poly Hydroxybutyrate resin, a biodegradable polymer.

According to the invention this is achieved by a plasticizer composition comprising
(i) PHB with the proviso that the PHB and its co-polymers of PHB are produced by a bio-polymer extraction process, characterized by the fact that the concentrated cellular material, previously dried or not, is mixed with an adequate solvent, specifically superior alcohol, preferably with a chain with more than 3 carbon atoms, or any other of its acetates, preferably isoamyl alcohol, amyl acetate, isoamyl acetate or the flusel oil as described by the Brazilian Patent PI 9302312-0 published in Apr. 30, 2002.
(ii) a) Fatty alcohols having a chain length from 6 to 30 carbon (C6-C30) with the proviso that the fatty alcohols can be saturated or olefinically unsaturated linear or branched and b) glycerol esters of fatty acids with 6 to 24 carbon atoms with the proviso that the fatty acids can be saturated or olefinically unsaturated, linear or branched.

Dry blend compositions of PHB and its co-polymer with fatty alcohols and glycerol esters used as plasticizer, are easily prepared by mixing the PHB and its co-polymers in a dry blend mixer at 90° C. for 5 minutes with slow addition of the plasticizers under mixing.

In one embodiment the plasticizer compositions of the invention contain compounds (i) and (ii) in an amount that the weight ratio of compounds (i) and (ii) is within the range of 95:5 and 50:50 and specially within the range of 90:10 and 75:25. The invention also relates to the use of compositions comprising the plasticizers (ii) with the proviso that they are composed of:
a)—Fatty alcohols having a chain length from 6 to 30 carbon (C6-C30), saturated or olefinically unsaturated, linear or branched,
b)—Glycerol esters of fatty acids with 6 to 24 carbon atoms with the proviso that the fatty acids can be saturated or olefinically unsaturated, linear or branched.

As also stated above the compositions preferably contain compounds (a) and (b) in an amount that the weight ratio of compounds (a) and (b) is within the range of 100:0 or 95:5 or 75:25 and 50:50 and especially within the range of 100:0 and 75:25. Those plasticizer compositions, which exclusively contain compounds (a) and (b), are preferred.

The PHB and its co-polymers provided for this invention have a Mw from 300,000 to 1,000,000 with the general formula:

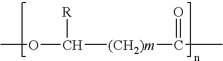

where R is an alkyl group of variable length m and n are integral numbers, and for PHB and its co-polymers R and m have the following values:
PHB: R═CH₃, m=1
PHB-V: R═CH₃ or CH₃—CH₂—, m=1
P4HB: R═H, m=2
P3HB4HB: R═H or CH₃, m=1 or 2
PHHx: R═CH₃—CH₂—CH₂—, m=1.

The preferred polymer used in accordance with this invention is a pure PHB with a molecular weight of 400,000 to 800,000.

The PHB and its co-polymers, according to this invention, come from a process (Brazilian Patent PI 9302312-0) which utilizes a solvent extraction process without the use of halogenated solvents.

The extraction process utilizes superior alcohols with chain length greater than 3 carbon atoms or the acetate derivatives Preferably isoamyl alcohol (3-methyl-1-butanol), amyl acetate and fusel oil or a mix of superior alcohols as a by product from an alcoholic fermentation process where the main component is isoamyl alcohol are used.

The process can be performed in a continuous or intermittent way and, in both cases, the cells containing the bio-polymer are processed by a single solvent, by what is characterized as a single stage process.

In this process, the concentrated cellular material, previously dried or not, is submitted to extraction with an adequate solvent, superior alcohol and/or its ester. After that, the cellular residue is separated by conventional mechanical techniques that can be deposition, flotation, filtering, centrifuging or also a combination of these methods, resulting in a cake and a solution containing the polymer. The latter is submitted to a crystallization stage that precipitates the polymer from the solvent without an agent that prevents dissolution. Crystallization may occur due to the increased concentration of the polymer in the solution, by removing the solvent (for example, evaporation), associated or not with the lowering of the temperature of the solution. In both cases, the polymer will solidify in the solution without the addition of a dissolving prevention agent and, then, it may be recovered from the solution by conventional mechanical separation (as mentioned above). Therefore, the separated solution may be directly recycled to the extraction stage.

The drying and extraction of the polymer can be done in a single stage if an adequate solvent is chosen, which is not or partially not soluble in water, as, for example isoamyl alcohol; water can be removed by distilling the mixture at its boiling point during the extraction. The distilled material can then be cooled forming two phases. The aqueous phase is discarded and the solvent returns directly to the extraction process.

In order to operate according to the system above, appropriate pressure and temperature conditions must be chosen in order to prevent the thermal decomposition of the polymer.

In order to increase the grain size and make crystallization easier, nucleating agents may be added.

The temperature range that is more adequate for polymer extraction is usually above 40° C. and the solvent boiling point (in the case of dry cells), or at the aqueous mixture boiling point (in the case of humid cells).

Once the hot dissolving is performed, the product precipitation occurs due to the cooling of the solution to ambient temperature. This cooling may eventually be preceded by an impurity purging.

The heating; cooling and purging operations are performed in the same vessel, or in two vessels placed in series, featuring devices to control the system's temperature. The vessels can also be equipped with a stirring system to accelerate the extraction and a system of flow-directing plates to enhance deposition. Alternatively, the cell suspension in the solvent may be heated in continuous flow through heat exchangers and, after that, transferred to a cooling and deposition vessel.

The quantity of solvent employed depends on the bio-polymer content in the cells and on the extraction time. The ratio between the solvent mass and the mass of the cells varies between 2.5 and 200, preferably between 10 and 150.

It is also an object of this invention to provide the use of a thermal stabilization system, constituted by: a primary antioxidant such as a hindered phenol (in an amount of 0.02% and 0.5%—% in mass concerning the totality of the PHB and the plasticizers); a secondary antioxidant such as an organic phosphite (in content of 0.02% and 0.5%—% in mass concerning the totality of the PHB and the plasticizer); a thermal stabilizer such as lactone (in content of 0.02% and 0.5%—% in mass concerning the totality of the PHB and the plasticizer).

It is also another object of this invention to provide the use of the sorbitol and sodium benzoate as nucleants. These nucleants are used for the thermodynamic and kinetic process controls of the PHB crystallization (nucleating and growth) of polymeric compositions. In accordance with crystalline morphology and with the degree of crystallinity desired the nucleant content must be varied with the cooling gradient imposed to the polymeric material during its final stage process.

The invention also relates to the use of fillers in the plasticizer compositions with the proviso that the fillers can be comprised of starch, wood powder, cane bagasse fibers, rice pod fibers and sisal fibers. These fillers are used to meet the specific process-structure-properties-cost relationship, for a specific product made with a polymeric composition based in PHB/plasticizer/additives.

Another embodiment of the invention is the use of the claimed composition as injection molding pieces and/or as films for packaging.

### EXAMPLES

A technical study was made with pure PHB and 6 different plasticizer compositions. Dry blend mixtures of PHB and the plasticizer compositions were produced by mixing them in a Mixer at 100° C. to 110° C. for 5 minutes and 5 minutes of cooling to 50° C.

The dry blend was palletized by extrusion and the test bodies were produced by injection molding as follow:

### Extrusion:

Co-Rotational double screw extruder—Werner & Pfleiderer ZSK-30 (30 mm)

Conditions:

| Samples | Temperature (° C.) | | | | | | | Speed |
|---|---|---|---|---|---|---|---|---|
| Zones | C1 | C2 | C3 | C4 | C5 | Matrix | Melt | (rpm) |
| PHB Pure | 128 | 132 | 154 | 140 | 150 | 152 | 152 | 140 |
| PHB/ | 130 | 137 | 138 | 140 | 148 | 148 | 154 | 150 |
| Plasticizer | | | | | | | | |
| (80/20) | | | | | | | | |
| PHB/ | 130 | 135 | 135 | 140 | 145 | 145 | 152 | 150 |
| Plasticizer | | | | | | | | |
| (70/30) | | | | | | | | |
| PHB/ | 120 | 135 | 135 | 140 | 145 | 145 | 150 | 150 |
| Plasticizer | | | | | | | | |
| (60/40) | | | | | | | | |

### Injection Molding:

Injection Machine-ARBURG 270 V-30 ton

Mold (for test bodies), ASTM D 638 (tensile Strength I) and ASTM D 256 (Impact Izod).

### Injection Molding Conditions:

| Temperature profile (° C.): | Pressure/time profiles |
|---|---|
| Zone 1: 152 | Pressure (bar): 400 |
| Zone 2: 156 | Pressurization (bar): 380 |
| Zone 3: 172 | Flow (cm³/s): 20 |
| Zone 4: 172 | Holding (bar): 300 |
| Zone 5: 170 | Time of holding (s): 12 |
| Mold (° C.): 35 | Back pressure (bar): 40 |
| Cooling time (s): 32 | Dosage Speed (mm/min): 12 |

Test Results

| | | Melt | Tensile | | Tensile | Izod | % | |
|---|---|---|---|---|---|---|---|---|
| | Density | Flow | strength | % | Modulus | Impact | cristalinity | TM |
| Samples | (g/cm3) | (g/10 min) | (MPa) | Elongation | (MPa) | (Notched) | (DSC) | (DSC) |
| PHB | 1.228 | 33.5 | 36.68 | 2.23 | 3.24 | 21.09 | 61.4 | 173.9 |
| (MW- | | | | | | | | |
| 380.000) | | | | | | | | |
| F2080 | 1.137 | 57.2 | 19.64 | 2.68 | 1.72 | 18.65 | 56.1 | 170.5 |
| F3080 | 1.088 | 95.6 | 15.25 | 3.77 | 1.13 | 21.09 | 55.3 | 166.7 |
| F4080 | 1.074 | 133.7 | 12.93 | 3.58 | 0.98 | 23.85 | 58.5 | 165.6 |
| F2100 | 1.126 | 49.6 | 19.40 | 3.22 | 1.59 | 19.23 | 57.0 | 168 |
| F3100 | 1.08 | 115 | 15.49 | 3.00 | 1.26 | 18.65 | 56.5 | 161.1 |
| F4100 | 1.042 | >150.0 | 10.27 | 2.89 | 0.82 | 25.13 | 58.8 | 164.8 |

Plasticized PHB Formulations

| | | Soy Bean Oil | |
|---|---|---|---|
| Plasticizer compositions | Oleyl alcohol | (Glycerol ester) | PHB |
| F2080 | 8% | 2% | 90% |
| F3080 | 16% | 4% | 80% |
| F4080 | 24% | 6% | 70% |
| F2100 | 10% | — | 90% |
| F3100 | 20% | — | 80% |
| F4100 | 30% | — | 70% |

What is claimed is:

1. A plasticized composition, comprising:
(i) at least one biopolymer selected from the group consisting of poly hydrobutyrate (PHB) and PHB copolymers; and
(ii) a plasticizer containing (a) a saturated or unsaturated, linear or branched C₆₋₃₀ fatty alcohol, and (b) a glycerol ester of a linear or branched, saturated or unsaturated C₆₋₂₄ fatty acid, wherein (a) and (b) are present in a ratio of 100:0 to 75:25 by weight.

2. The composition according to claim 1, wherein the ratio of component (i) to component (ii) is 90: 10 to 75:25 by weight.

3. The composition according to claim 1, further comprising a thermal stabilization system comprising: a primary antioxidant; a secondary antioxidant; and a thermal stabilizer.

4. The composition according to claim 3, wherein the primary antioxidant is a hindered phenol, the secondary antioxidant is an organic phosphite, and the thermal stabilizer is lactone.

5. The composition according to claim 1, further comprising a nucleant.

6. The composition according to claim 5, wherein the nucleant is selected from the group consisting of sorbitol and sodium benzoate.

7. The composition according to claim 1, further comprising a filler additive selected from the group consisting of starch, wood powder, cane bagasse fibers, rice pod fibers, sisal fibers, and mixtures thereof.

8. The composition according to claim 1, wherein (i) is defined according to the following formula: 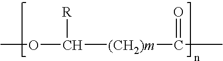
wherein R is an alkyl group of variable length, m and n are integral numbers, and wherein for each of the following PHB and PHB co-polymers, R and m have the following values: for PHB, R═CH₃ and m=1; for PHB-V, R═CH₃ or CH₃—CH₂— and m=1; for P4HB, R═H and m=2; for P3HB4HB, R═H or CH₃, and m=1 or 2; and for PHHx, R═CH₃—CH₂—CH₂— and m=1.

9. The composition according to claim 1, wherein the molecular weight of the PHB is within the range of 300,000 to 1,000,000.

10. The composition according to claim 1, incorporated into an injection molded article and/or a film for packaging.

11. The composition according to claim 1, wherein the at least one biopolymer is recovered from cells from a fermentation medium by a process which comprises:
(a) extracting the polymer from wet or dry cells with an extractant selected from the group consisting of alcohols with more than 3 carbon atoms, acetates of alcohols with more than three carbon atoms, fusel oil, and mixtures thereof,
(b) separating the extractant from the extracted cells; and
(c) crystallizing the biopolymer from the extractant.

12. The composition according to claim 11, wherein the extractant is isoamyl alcohol.

13. The composition according to claim 11, wherein the extractant is amyl acetate.

14. The composition according to claim 11, wherein the extractant is isoamyl acetate.

15. The composition according to claim 11, wherein the extractant is fusel oil.
